(19) United States
(12) Patent Application Publication
(10) Pub. No.: US 2015/0231611 A1
(43) Pub. Date: Aug. 20, 2015

(54) METHODS AND APPARATUSES FOR REGENERATING CATALYSTS FOR HYDROCARBON PRODUCTION

(71) Applicant: UOP LLC, Des Plaines, IL (US)

(72) Inventors: Christopher Lepine Standing, McCook, IL (US); Gregory J. Gajda, Mt. Prospect, IL (US); Bart Dziabala, Hickory Hills, IL (US); Robert James Schmidt, Barrington, IL (US)

(73) Assignee: UOP LLC, Des Plaines, IL (US)

(21) Appl. No.: 14/184,389

(22) Filed: Feb. 19, 2014

(51) Int. Cl. B01J 23/96; C07C 5/05

(52) U.S. Cl. CPC B01J 23/96; C07C 5/05

(57) ABSTRACT

Methods and apparatuses are provided for producing hydrocarbons. A method includes contacting an aromatic rich feed stream including diolefins with a catalyst in the presence of hydrogen to react the diolefins with the hydrogen to produce mono-olefins. A deposit forms on the catalyst during reaction. The deposit is removed from the catalyst with a solvent, where the solvent includes about 50 mass percent or more aromatic compounds.

### FIELD OF THE INVENTION

The present disclosure generally relates to methods and apparatuses for regenerating catalysts used for hydrocarbon production, and more particularly relates to methods and apparatuses for regenerating catalysts used for converting diolefins into mono-olefins.

### BACKGROUND

Pyrolysis gasoline is often obtained as a by-product from thermal cracking of various hydrocarbons. The pyrolysis gasoline often includes many aromatic compounds, as well as diolefins (hydrocarbons with two sets of double bonds), mono-olefins (hydrocarbons with one double bond), alkanes with no double bonds, and sulfur and nitrogen compounds. Depending on the feed source to the steam cracker pyrolysis gasoline may also contain metal contaminants. Pyrolysis gasoline can be used as a source for aromatic compounds, but the diolefins, mono-olefins, sulfur and nitrogen compounds need to be removed before the aromatic compounds can be recovered by various processes, such as solvent extraction.

The pyrolysis gasoline is often treated in a two-step process prior to separating and purifying the aromatic compounds. In the first step, diolefins and any alkynes are selectively hydrogenated to form mono-olefins and some paraffins. The first step is operated under moderate conditions with a selective catalyst such that primarily diolefins are reacted to mono-olefins. At the same time some of the mono-olefins are saturated and very few, if any, aromatic compounds are saturated. In the second step, the mono-olefins are saturated (hydrogenated) to form alkanes, and the nitrogen and sulfur compounds are removed. The second step is operated under more severe reaction conditions that would cause diolefins to polymerize and undesirably result in reactor pressure drop issues, so the first step is used to remove the more reactive diolefins prior to the second step. The first step is operated at moderate conditions with a selective catalyst, so diolefins are reacted to mono-olefins, but relatively few mono-olefins are saturated and aromatic compounds are essentially not saturated. The diolefins are far more reactive than the mono-olefins and aromatic species. The first step is often operated at a reactor inlet temperature of about 50 to about 150° C. with a delta temperature of up to about 20-50° C. across the reaction zone and a maximum outlet temperature of about 200 degrees centigrade (° C.) or less. The second step is often operated at an inlet temperature of about 250 to about 350° C. with about a 30-50° C. delta temperature across the reaction zone and a maximum outlet temperature of about 400° C. The diolefins and mono-olefins are hydrogenated in separate reactors, i.e. the first and second steps are conducted in separate reactors, to limit and control polymerization of the diolefins. Reducing mono-olefin hydrogenation reactions in the first stage limits excessive heat from the exothermic reaction that causes polymerization of diolefins.

A deposit of heavy polymerate gradually accumulates and deactivates the catalyst in the first step, so this deposit is periodically removed. The polymerate is currently removed with a hot, gaseous hydrogen strip with temperatures that can reach 370° C. Over time, this gaseous strip reduces the activity of the catalyst for the first stage. In many embodiments, the catalyst includes palladium (or other metals) on an alumina support, where the palladium is reacted with sulfur to reduce and control the catalytic activity. Without being bound to any particular theory, it is believed that the hot hydrogen strip tends to break the sulfur—metal bonds as well as agglomerate the palladium on the support. Agglomeration of the palladium changes the cluster size of the metal site, and therefore reduces the effectiveness and lifespan of the catalyst. The removal of sulfur from the catalyst alters the catalytic selectivity for hydrogenating diolefins. For example, testing has shown that a single catalyst regeneration with a hot hydrogen strip can reduce diolefin selectivity to olefins by about 17 weight percent.

Accordingly, it is desirable to develop methods and apparatuses for regenerating catalyst for hydrocarbon production using mild conditions. In addition, it is desirable to develop methods and apparatuses for removing deposits from catalyst without stripping sulfur from the catalyst. Furthermore, other desirable features and characteristics of the present embodiment will become apparent from the subsequent detailed description and the appended claims, taken in conjunction with the accompanying drawings and this background.

### SUMMARY OF THE INVENTION

Methods and apparatuses for producing hydrocarbons are provided. In an exemplary embodiment, a method includes contacting an aromatic rich feed stream including diolefins with a catalyst in the presence of hydrogen to react the diolefins with the hydrogen to produce mono-olefins. A deposit forms on the catalyst during the reaction. The deposit is removed from the catalyst with a solvent, where the solvent includes about 30 mass percent or more aromatic compounds.

In accordance with another exemplary embodiment, a method for regenerating a catalyst is provided. The catalyst is contacted with a solvent that includes 30 mass percent or more aromatic compounds. The catalyst includes palladium on a support, and a heavy polymerate contacts the catalyst. The heavy polymerate is removed from the catalyst with the solvent to produce a spent solvent, and the spent solvent is removed from the catalyst.

In accordance with a further exemplary embodiment, an apparatus for selectively hydrogenating diolefins is provided. The apparatus includes a reactor configured to contain a catalyst. A fractionation zone is coupled to the reactor, and a second stage reactor is coupled to the fractionation zone. A spent solvent line extends from the reactor to the fractionation zone, and a solvent stream line extends from the fractionation zone to the reactor.

### BRIEF DESCRIPTION OF THE DRAWINGS

The present embodiment will hereinafter be described in conjunction with the following drawing figures, wherein like numerals denote like elements, and wherein:

FIG. 1 is a schematic diagram of an exemplary embodiment of an apparatus and a method for producing hydrocarbons; and

FIG. 2 is a schematic diagram of an exemplary embodiment of an apparatus and method for regenerating a catalyst.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The various embodiments described herein relate to methods and apparatuses for producing hydrocarbons and regenerating catalysts with a solvent wash. Diolefins in an aromatic rich feed stream are reacted with hydrogen in the presence of a catalyst to produce mono-olefins in a reactor effluent stream, and a deposit gradually builds up on the catalyst. The reactor effluent stream is fractionated in a fractionation zone to produce a C5-stream, a C6-8 stream, and a C9+ stream, where the letter “C” represents carbon, and the following number represents the number of carbon atoms present in the molecule. Aromatic compounds have at least 6 carbons, so the aromatic compounds are present in the C6-8 and the C9+ streams, but not in the C5-stream. The catalyst is periodically regenerated by washing with a solvent that has a high concentration of aromatic compounds, where the temperature of the wash is greater than the reaction temperature. In some embodiments, the C6-8 stream is used as the solvent, and the deposit and solvent can be separated in the fractionation zone so the solvent can be recirculated through the catalyst to dissolve more polymerate, or otherwise used. The deposit can then be transferred and used in other processes. The solvent is used in a liquid state, so moderate temperatures and pressures can be used to minimize degradation of the catalyst.

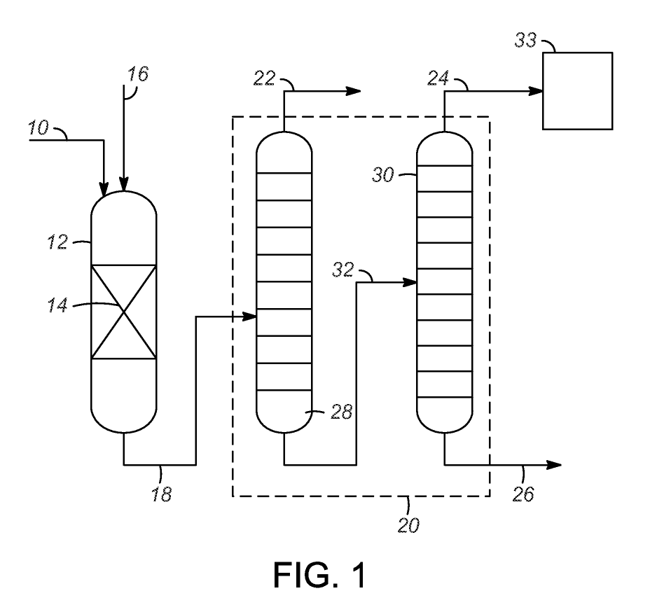

Reference is made to the exemplary embodiment illustrated in FIG. 1. An aromatic rich feed stream 10 is fed into a reactor 12, where the reactor 12 is configured to contain a catalyst 14. The aromatic rich feed stream 10 primarily includes C4-10 hydrocarbons, such that about 90 mass percent or more of the aromatic rich feed stream 10 is hydrocarbons with 4 to 10 carbon atoms. The aromatic rich feed stream 10 includes about 30 mass percent or more aromatic compounds, so it is rich in aromatic compounds. The aromatic rich feed stream 10 may be a pyrolysis gasoline produced by steam cracking, but the aromatic rich feed stream 10 may also include other sources, such as a coke oven light oil.

The aromatic rich feed stream 10 includes aromatic compounds, and often includes about 30 to about 90 mass percent aromatic compounds. The aromatic rich feed stream 10 also includes diolefins and mono-olefins, saturates (hydrocarbons without double or triple bonds between adjacent carbon atoms) sulfur and/or nitrogen compounds, and may include some alkynes, and metal contaminants. The feedstock and operating conditions in a steam cracker that produces pyrolysis gasoline varies widely, so the components of the aromatic rich feed stream 10 also vary widely. As an example, one pyrolysis gasoline aromatic rich feed stream 10 included about 13 mass percent C5-compounds, about 59 mass percent C6-8 compounds, and about 28 mass percent C9+ compounds, where about 74 mass percent of the entire stream was aromatic compounds, 14 mass percent was diolefins, about 6 mass percent was mono-olefins, and about 5 mass percent was saturates. In this example the feed contained about 225 wppm sulfur and about 17 wppm nitrogen. As mentioned above, the concentration of the various components in the aromatic rich feed stream 10 can vary significantly from the example described above.

In the embodiment illustrated in FIG. 1, a hydrogen supply stream 16 is coupled to the reactor 12 and provides hydrogen gas. The aromatic rich feed stream 10 contacts the catalyst 14 in the reactor 12 in the presence of hydrogen, where the diolefins are catalytically hydrogenated to form mono-olefins. Alkynes are also reacted to form mono-olefins, and some olefins are reacted to form saturates. Aromatic compounds include more than 2 sets of double bonds, but aromatic compounds are more stable than diolefins so relatively few to none of the aromatic compounds in the aromatic rich feed stream 10 are hydrogenated in the reactor 12. In an exemplary embodiment, about 1 mass percent or less of the aromatic compounds in the aromatic rich feed stream 10 are hydrogenated in the reactor 12. Low temperatures are used in the reactor 12, which produces mild reaction conditions, with temperatures ranging from about 40 to about 200° C. In an exemplary embodiment, the inlet temperature is about 40 to about 60° C., and the outlet temperature is about 120 to about 150° C. The reaction pressure can vary, such as from about 2,000 kilopascals (kPa) to about 7,000 kPa. The liquid hourly space velocity (LHSV) of the aromatic rich feed stream 10 can also vary over a wide range. Typically, the LHSV will be in the range of about 0.5 to about 30 liters of aromatic rich feed stream 10 per liter of catalyst per hour. The temperature, pressure, and LHSV variables are controlled to avoid significant hydrogenation of mono-olefins, if present.

The catalyst 14 selectively catalyzes hydrogenation of diolefins and alkynes to produce mono-olefins and some olefins to saturates, but has little catalytic activity for hydrogenation of aromatic compounds at the reaction conditions in the reactor 12. In an exemplary embodiment, the catalyst 14 includes a metal from group 10 of the periodic table of elements (nickel, palladium, and platinum), and a support. The group 10 metal can be in one of several forms, such as in the metal form, oxide form, or sulfide form. In some embodiments, the catalyst 14 also includes one or more other metals or metal compounds, such as a metal or metal compound from groups 8 and/or 9 and/or 11 of the periodic table of elements (iron, ruthenium, osmium, cobalt, rhodium, iridium, copper, silver, and gold), and/or one or more alkali metals which may include an acidity modifier. Any of the metals may be sulfided, where the metal is reacted with sulfur to form a metal sulfide. The sulfided metal form tends to reduce the activity of the catalytic metal and improve the catalyst's selectively to hydrogenate diolefins over mono-olefins. In some embodiments, sulfur compounds in the aromatic rich feed stream 10 maintain the catalyst 14 in a sulfided state. The support can be any of a wide variety of materials, such as aluminum oxide, silicon oxide, titanium oxide, zirconium oxide, aluminum phosphate, scandium oxide, yttrium oxide, magnesium oxide, silica, aluminosilicates (clays, zeolites), activated carbon, and combinations thereof. In some embodiments, the support includes one or more aluminum oxide (alumina), such as alpha-alumina, theta-alumina, gamma-alumina, boehmite, diaspore, bayerite and/or pseudoboehmite. The support is alkali treated in some embodiments to remove acidity. Particles of the catalyst 14 can have many shapes, including but not limited to spherical, cylindrical, granular, and trilobal, and the catalyst particle size can vary widely as well, such as from an average size of about 0.1 to about 100 millimeters (mm), as a length or diameter.

In the embodiment illustrated in FIG. 1, a reactor effluent stream 18 exits the reactor 12, where the reactor effluent stream 18 primarily includes mono-olefins, alkanes, and aromatic compounds. There are very few if any diolefins and alkynes in the reactor effluent stream 18, such as less than about 1 mass percent. The reactor effluent stream 18 may be fractionated in a fractionation zone 20 to produce various fractions for further processing or use, so the reactor 12 is coupled to the fractionation zone 20. In an exemplary embodiment, the reactor effluent stream 18 is fractionated to form a C4-5 stream 22, a C6-8 stream 24, and a C9+ stream 26. The fractionation zone 20 includes a depentanizer 28 and a deoctanizer 30 in an exemplary embodiment, but the fractionation zone 20 may have only one fractionation column in other embodiments, or more than 2 fractionation columns in other embodiments. The depentanizer 28 is illustrated in front of the deoctanizer 30 in FIG. 1, but the order can be reversed in other embodiments. In an exemplary embodiment, the depentanizer 28 has an overhead pressure of about 300 to about 400 kPa (absolute) and a temperature of about 30 to about 50° C., with a bottoms pressure of about 400 to about 500 kPa (absolute) and a temperature of about 100 to about 150° C. The deoctanizer 30, in an exemplary embodiment, has an overhead pressure of about 5 to about 50 kPa (absolute) and a temperature of about 20 to about 50° C., with a bottoms pressure of about 30 to about 80 kPa (absolute) and a temperature of about 100 to about 200° C.

In an exemplary embodiment, the C4-5 stream 22 exits at the overhead of the depentanizer 28, and a C6-9+ stream 32 exits at the bottoms. The C6-9+ stream 32 is then fractionated in the deoctanizer 30, where the C6-8 stream 24 exits at the overheads and the C9+ stream 26 exits at the bottoms. The C6-8 stream 24 includes most of the aromatic compounds, as described above, so the concentration of aromatic compounds is higher than in the aromatic rich feed stream 10 or the reactor effluent stream 18. In an exemplary embodiment, the C6-8 stream 24 includes about 50 mass percent or more aromatic compounds. In an exemplary embodiment, the fractionation zone 20 is coupled to a second stage reactor 33, and the C6-8 stream 24 is transferred to the second stage reactor 33 for further processing, such as for removal of sulfur and nitrogen compounds and hydrogenation of mono-olefins. The C4-5 stream 22 and the C9+ stream 26 may be transferred to other areas for blending, further processing, or other uses.

The catalytic reaction of diolefins with hydrogen produces deposits that adhere to the catalyst 14, and the deposits may adhere to the walls of the reactor 12 as well. Without being bound to any particular theory, it is believed that the catalytic reaction of diolefins with other diolefins produces a heavy polymerate, which includes a polymer formed from the diolefins or other compounds in the aromatic rich feed stream 10. The deposits form gradually, and deactivate the catalyst 14 as the deposits accumulate on the catalyst 14. The deposits may not be soluble in the aromatic rich feed stream 10 at the temperature in the reactor 12 during the diolefin hydrogenation reaction, so the deposits accumulate. At some point, the activity of the catalyst 14 is reduced to the point where the catalyst 14 needs to be regenerated. For example, the catalyst 14 may be regenerated when the catalyst activity degrades to the point where a temperature of the reactor effluent stream 18 exiting the reactor 12 exceeds a set point, such as about 110° C. Alternatively, the catalyst 14 may be regenerated when the pressure drop across the reactor 12 increases beyond a set limit. Other criteria can also be used to determine when to regenerate the catalyst 14.

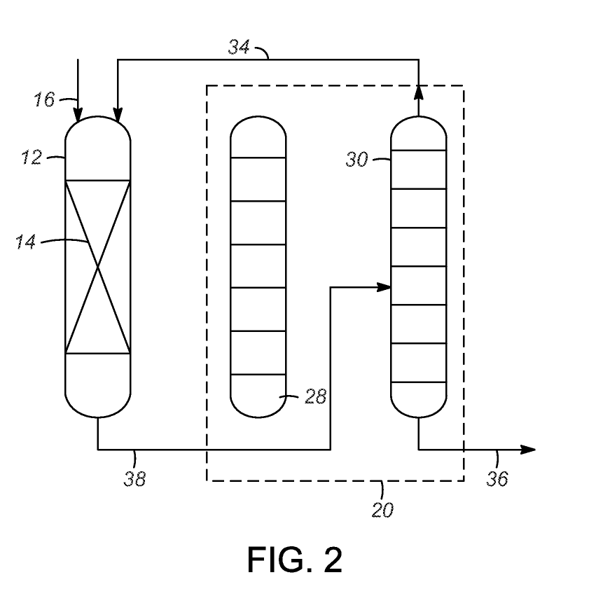

Referring now to FIG. 2, with continuing reference to FIG. 1, flow of the aromatic rich feed stream 10 to the reactor 12 is stopped while the catalyst 14 is regenerated. A solvent stream 34 introduces a solvent to the reactor, where the solvent contacts the catalyst 14. In some embodiments, the solvent has about 30 mass percent or more aromatic compounds. The solvent flows over and through the catalyst 14 in a liquid state and is maintained at a temperature of about 100 to about 230° C. in some embodiments, or about 150 to about 230° C. in other embodiments, and at a pressure sufficient to keep the solvent in the liquid state. In some embodiments, the solvent is maintained at a temperature above the temperature in the reactor 12 while hydrogenating diolefins. Hydrogen or other gases can be used to control the pressure in the reactor 12 during the regeneration process, but the solvent is used in a liquid state so the concentration of hydrogen in the solvent is relatively low. The relatively low temperature of the solvent (relative to hydrogen stripping temperatures) does not cause significant agglomeration of the metal on the catalyst 14, so the catalyst 14 remains selective. However, the higher temperature of the solvent, relative to the diolefin hydrogenation reaction temperature, increases the solubility of the deposits in the solvent. Also, the higher solvent temperature, relative to the diolefin hydrogenation reaction temperature, may cause trans-alkylation where some of the deposits react with lower molecular weight aromatic compounds to reduce the molecular weight of the deposit compounds while increasing the molecular weight of the aromatic solvent compounds. This increases the solubility of the deposits, and may increase the concentration and yield of heavier and alkylated aromatics as carbon atoms are transferred from molecules in the deposit (which are heavier than xylene) to aromatic solvent compounds such as benzene or toluene (which are lighter than xylene). As such, the deposits may be removed from the catalyst 14 by different mechanisms including dissolution and reaction.

The solvent is passed through the catalyst 14 for a sufficient period of time to remove most or all of the deposits from the catalyst 14, as well the walls of the reactor 12, and the solvent and deposits are separated from the catalyst 14 and exit the reactor 12 as a spent solvent stream 38. In an exemplary embodiment, the solvent flows through the catalyst 14 for about 12 to about 36 hours to regenerate the catalyst 14, but other periods of time are also possible. In some embodiments, the regeneration period is determined by the amount of polymer collected in the spent solvent stream 38. Higher solvent temperatures allow for shorter regeneration periods, and lower solvent temperatures may reduce or minimize sulfur loss from the catalyst 14.

In an exemplary embodiment, the solvent may include sulfur compounds, such as mercaptans or sulfides, so the catalyst remains sulfided during regeneration. Alternatively, the solvent temperature and the time period for the regeneration process are sufficiently limited to prevent significant stripping of sulfur from the catalyst 14, so the catalyst 14 remains sulfided during and after the regeneration process. One mechanism that can strip sulfur from the catalyst includes reacting the sulfur with hydrogen to produce hydrogen sulfide. In some embodiments, the hydrogen concentration in the solvent is limited to about 1 mass percent or less to help minimize sulfur stripping from the catalyst 14.

In an exemplary embodiment, the C6-8 stream 24 is used as the solvent. The C6-8 stream 24 has about 30 mass percent or more aromatics in some embodiments, and includes sulfur compounds present in the aromatic rich feed stream 10. The C6-8 stream 24 is readily available for the process, and can be used with relatively few equipment changes because the process flow is similar to when the aromatic rich feed stream 10 is being fed to the reactor 12. The C6-8 stream 24 may be temporarily stored in a holding tank (not illustrated) before being introduced to the second stage reactor 33, or the C6-8 stream 24 can be collected in temporary storage (not illustrated) prior to beginning the solvent wash and catalyst regeneration process.

In an exemplary embodiment, the solvent and the deposit in the spent solvent stream 38 are separated by fractionation after exiting the reactor 12 to produce a deposits stream 36 with the deposit and to regenerate the solvent. A spent solvent line may direct the spent solvent stream 38 from the reactor 12 to the deoctanizer 30, such that the depentanizer 28 is by-passed. The deoctanizer 30 can be run at standard operating conditions, as described above, to produce the solvent stream 34 at the overheads and the deposits stream 36 at the bottoms. The deposits are liquid at the temperature and pressure of the deoctanizer bottoms (about 100 to about 200° C. at about 30 to about 80 kPa, as described above), so the deposits stream 36 flows out of the deoctanizer 30 as a liquid. The deposits stream 36 can then be recycled back to the steam cracker, processed in another downstream hydroprocessing unit, or otherwise used or disposed of Many oil refineries have common processes for recycling streams similar to the deposits stream 36, so the deposits stream 36 could be added to such existing systems. The solvent stream 34 produced at the overheads of the deoctanizer 30 can then be recycled to the reactor 12 through a solvent stream line extending from the deoctanizer 30 to the reactor 12. In this manner, the solvent can be repeatedly cycled through the catalyst 14 and the deoctanizer 30 to wash and regenerate the catalyst 14. When the regeneration process is complete, the solvent stream 34 can be combined with the C6-8 stream 24 produced while processing the aromatic rich feed stream 10.

In an alternate embodiment not illustrated in the FIGS., the solvent stream is a stream other than the C6-8 stream 24, and is added to the reactor from a separate line. The solvent stream may include about 30 mass percent or more aromatic compounds in some embodiments. The spent solvent stream 38 can then be fractionated in the fractionation zone 20, as described above in the context of FIG. 2, to separate the deposits and prepare the solvent stream 34 for further use. In this embodiment, the operating conditions of the fractionation zone 20 may be modified if the composition of the spent solvent stream 38 is significantly different than the process streams fractionated during reaction of the diolefins, as understood by those skilled in the art. The solvent stream 34 can be discharged, recycled, otherwise used, or disposed of after the catalyst regeneration process is complete.

Suitable solvent streams 34 are available in many oil refineries, such as from an aromatic complex or separation process. In one exemplary embodiment, a product from the second stage reactor 33 is used as the solvent stream. This stream will typically range from about 30 to over 70% mass percent aromatics and is a high quality stream that is subsequently used as a feed to a downstream aromatics complex or separation process, such as recovery of xylenes, toluene, and/or benzene. In some embodiments, the somewhat higher temperatures during the solvent wash, as compared to the diolefin hydrogenation reaction, may cause some limited formation of deposits from diolefins or mono-olefins. Essentially all of the diolefins and mono-olefins are saturated in the product of the second stage reactor 33, and the low concentration of olefins may decrease the required wash time by reducing the formation of additional deposits during the wash. In this stream, the sulfur and nitrogen levels have been reduced, such as to about 0.5 parts per million by weight or less. If the solvent stream is low in sulfur, such as the product of the second stage reactor 33, sulfur compounds may be added during the solvent wash, such as with a hydrogen disulfide or mercaptan injection.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

1. A method of producing hydrocarbon compounds comprising:
contacting an aromatic rich feed stream comprising diolefins with a catalyst in the presence of hydrogen to react the diolefins with the hydrogen to produce mono-olefins, and wherein a deposit forms on the catalyst during the reaction; and
removing the deposit from the catalyst with a solvent, wherein the solvent comprises about 30 mass percent or more aromatic compounds.

2. The method of claim 1 wherein dissolving the deposit with the solvent further comprises dissolving the deposit with the solvent wherein the solvent comprises a reactor effluent stream, and wherein contacting the aromatic rich feed stream with the catalyst in the presence of hydrogen produces the reactor effluent stream.

3. The method of claim 2 further comprising:
fractionating the reactor effluent stream to produce the solvent.

4. The method of claim 1 wherein contacting the aromatic rich feed stream with the catalyst comprises contacting the aromatic rich feed stream with the catalyst comprising a metal from group 10 of the periodic table, and wherein the catalyst further comprises a support selected from one or more of aluminum oxide, silicon oxide, titanium oxide, and zirconium oxide.

5. The method of claim 1 wherein contacting the aromatic rich feed stream with the catalyst comprises contacting the aromatic rich feed stream with the catalyst comprising palladium on an aluminum oxide support.

6. The method of claim 5 wherein contacting the aromatic rich feed stream with the catalyst comprises contacting the aromatic rich feed stream with the catalyst wherein the catalyst is sulfided.

7. The method of claim 1 wherein contacting the aromatic rich feed stream with the catalyst in the presence of hydrogen produces the deposit comprising a heavy polymerate.

8. The method of claim 1 further comprising:
maintaining a temperature of the solvent from about 150 degrees centigrade to about 230 degrees centigrade while dissolving the deposit.

9. The method of claim 1 further comprising:
limiting hydrogen in the solvent to about 1 mass percent or less.

10. The method of claim 1 further comprising:
terminating the contact of the aromatic rich feed stream with the catalyst prior to dissolving the deposit with the solvent.

11. The method of claim 10 further comprising:
fractionating a spent solvent to produce a deposits stream and the solvent, wherein the spent solvent comprises the solvent and the deposit.

12. The method of claim 1 further comprising:
adding a sulfur compound to the solvent while dissolving the deposit with the solvent.

13. A method of regenerating a catalyst comprising:
contacting the catalyst with a solvent, wherein the catalyst comprises palladium on a support, wherein a heavy polymerate contacts the catalyst, and wherein the solvent comprises 30 mass percent or more aromatic compounds;
removing the heavy polymerate from the catalyst with the solvent to produce a spent solvent; and
removing the spent solvent from the catalyst.

14. The method of claim 13 further comprising:
maintaining a temperature of the solvent from about 150 degrees centigrade to about 230 degrees centigrade while the solvent contacts the catalyst.

15. The method of claim 13 wherein contacting the catalyst with the solvent further comprises contacting the catalyst with the solvent wherein the support comprises one or more of aluminum oxide, silicon oxide, titanium oxide, and zirconium oxide.

16. The method of claim 13 wherein contacting the catalyst with the solvent comprises contacting the catalyst with the solvent wherein the solvent comprises about 1 mass percent hydrogen or less.

17. The method of claim 13 wherein contacting the catalyst with the solvent comprises contacting the catalyst with the solvent wherein the solvent comprises a sulfur compound.

18. The method of claim 13 further comprising:
fractionating the spent solvent to produce a deposits stream and a solvent stream, wherein the solvent stream comprises the solvent.

19. The method of claim 18 wherein contacting the catalyst with the solvent further comprises contacting the catalyst with the solvent stream produced by fractionating the spent solvent.

20. An apparatus for selective hydrogenation of diolefins comprising:
a reactor configured to contain a catalyst;
a fractionation zone coupled to the reactor;
a second stage reactor coupled to the fractionation zone;
a spent solvent line extending from the reactor to the fractionation zone; and
a solvent stream line extending from the fractionation zone to the reactor.
